(19) United States
(12) Patent Application Publication
(10) Pub. No.: US 2005/0220786 A1
(43) Pub. Date: Oct. 6, 2005

(54) Lyophilised preparation comprising antibodies against the efg receptor

(75) Inventors: Hanns-Christian Mahler, Wiesbaden (DE); Hans-Peter Zobel, Florsheim (DE); Robert Mueller, Darmstadt (DE); Christiane Bachmann, Goldbach (DE); Udo Haas, Darmstadt (DE); Ulrike Altenburger, Weil am Rhein (DE); Ludwig Krueger, Aschaffenburg (DE); Margarete Krueger, Aschaffenburg (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH, Darmstadt State (DE)

(21) Appl. No.: 10/499,569

(22) PCT Filed: Nov. 25, 2002
(86) PCT No.: PCT/EP02/13223

§ 371 Date: Mar. 10, 2005

(30) Foreign Application Priority Data
Dec. 21, 2001 (DE) 101 63 459.5

(51) Int. Cl. A61K 39/395; A61K 31/7008; A61K 31/7012

(52) U.S. Cl. 424/141.1; 514/53; 514/62

(57) ABSTRACT

The invention relates to a lyophilised pharmaceutical preparation comprising an antibody against the endothelial growth factor receptor (EGF receptor). The preparation has increased storage stability, even at elevated temperatures, and, after reconstitution, can be used parenterally for the treatment of tumours.

The present invention relates to a stable lyophilised pharmaceutical preparation comprising an antibody which is directed against the epidermal growth factor receptor (EGFR), and to the preparation thereof.

Various in vitro and in vivo studies have shown that blockage of the EGFR by antibodies acts against tumours at various levels, for example by inhibiting cancer cell proliferation, reducing tumour-mediated angiogenesis, induction of cancer cell apoptosis and increasing the toxic effects of radiotherapy and conventional chemotherapy.

MAB c225 (Cetuximab®) is a clinically proven antibody which binds to the EGF receptor. Cetuximab® is a chimeric antibody whose variable regions are of murine origin and whose constant regions are of human origin, and was described for the first time by Naramura et al., Cancer Immunol. Immunotherapy 1993, 37: 343-349 and in WO 96/40210 A1.

MAB 425 is an originally murine antibody which is overexpressed in tumour cells and is directed against the EGFR, in particular of A431 carcinoma cells. Its humanised and chimeric forms are disclosed, for example, in EP 0 531 472 A1; Kettleborough et al., Protein Engineering 1991, 4: 773-783; Bier et al., Cancer Chemother. Pharmacol. 2001, 47: 519-524; Bier et al., Cancer Immunol. Immunother. 1998, 46: 167-173. EMD 72000 here is an antibody (h425) which is in clinical phase I/II and whose constant region is composed of a κ and a human γ-1 chain.

Human anti-EGFR antibodies can be provided by XenoMouse technology, as described in WO 91/10741 A1, WO 94/02602 A1 and WO 96/33735 A1. A specific antibody which has been produced by this technology and is currently undergoing clinical trials is ABX-EGF (Abgenix, Crit. Rev. Oncol. Hematol., 2001, 38: 17-23; Cancer Research 1999, 59: 1236-43).

Further antibodies directed against the EGFR are described, for example, in EP 0 586 002 B1 and in J. Natl. Cancer Inst., 1993, 85: 27-33 (MAB 528).

Like other antibodies, EGFR antibodies are also applied parenterally as a solution for therapeutic use. A particular problem of solutions containing these antibodies is their tendency toward aggregation and the formation of protein multimers. In the case of reducible multimers, this can be attributed to unintentional intermolecular disulfide bridge formation through interaction between approaching moieties. Hydrophobic interactions and the consequent formation of non-reducible multimers are also possible. Furthermore, deamidation reactions, which subsequently result in protein degradation reactions, also occur. The denaturing reactions described occur, in particular, on storage at elevated temperature or during shear stresses, as occur, for example, during transport. In overall terms, liquid preparations are therefore of lower suitability as medicament form for broad use.

A customary process for the stabilisation of antibodies is freeze-drying of solutions containing antibodies and auxiliaries. Removal of the water reduces the formation of decomposition products and aggregates. (Hsu et al., Dev. Biol. Stand. 1991, 74: 255-267 and Pikal et al., Dev. Biol. Stand. 1991, 74: 21-27).

WO 93/00807 A1 describes lyophilised preparations of proteins which, for stabilisation, comprise polyethylene glycols and a sugar. However, polyethylene glycols are toxicologically dubious and should therefore be avoided in medicaments if possible, in particular if they are intended for parenteral administration.

WO 98/22136 A2 discloses a lyophilised preparation comprising an antibody, a sugar or amino sugar, an amino acid and a surfactant. Although the preparation is claimed for antibodies in general, the only preparations disclosed as working example are those comprising monoclonal antibodies directed against the hepatitis B virus (AK HBV), and in each case a preparation comprising an antibody against L-selectin (anti-L-selectin) and an antibody against the anti-L nerve growth factor receptor (anti-LNGFR). Whereas the preparations comprising AK HBV and anti-L-selectin were prepared from solutions having a maximum antibody concentration of 8 mg/ml and 7 mg/ml respectively, the preparation comprising the antibody directed against growth factor, anti-LNGFR, was prepared from a solution comprising only 0.25 mg/ml of antibody with an otherwise identical qualitative and quantitative composition of the auxiliaries. Although the preparation comprising anti-LNGFR thus has a more than 20-fold lower antibody content and a correspondingly lower amount of degradation products can therefore also be expected, no stability data are disclosed, in contrast to the preparations comprising the other antibodies.

The object of the present invention was to provide a stabilised preparation for antibodies directed against the EGFR. The preparation should not comprise any toxicologically unacceptable auxiliaries, should be stable for a relatively long time under increased stress conditions, such as elevated temperature and atmospheric humidity, and should be reconstitutable with an aqueous solvent to give a ready-to-use solution with a high active-ingredient content.

Surprisingly, a preparation which meets these requirements has been provided by freeze-drying an aqueous solution which, besides one of these anti-EGFR antibodies, also comprises a sugar or an amino sugar, an amino acid and a surfactant. The present invention therefore relates to a stable lyophilised preparation of mono- or polyclonal antibodies comprising a sugar or an amino sugar, an amino acid and a surfactant, characterised in that the antibody is an antibody directed against the epidermal growth factor receptor (EGFR).

The antibody that may be present is any antibody that is directed against epidermal growth factor, in particular the murine, humanised or chimeric antibodies mentioned at the outset and the human anti-EGFR antibodies which have been and can be prepared by means of the said XenoMouse technology. The anti-EGFR antibody present in the preparation according to the invention is preferably Cetuximab® or EMD 72000 or one of the murine, humanised or chimeric antibody analogues corresponding thereto. Particular preference is given to preparations which comprise Cetuximab® or EMD 72000 as antibody.

The preparation according to the invention is physiologically well tolerated, can be prepared easily, can be dispensed precisely and is stable with respect to assay, decomposition products and aggregates over the duration of storage and during repeated freezing and thawing processes. It is stable on storage over a period of at least three months to a period of from one to two years at refrigerator temperature (2-8° C.) and at room temperature (23-27° C., 60% relative atmospheric humidity (RAH)). Surprisingly, the preparation according to the invention is also stable on storage over the said period at elevated temperatures and higher atmospheric humidity levels, for example at a temperature of 40° C. and 75% relative atmospheric humidity.

The lyophilised preparation can be reconstituted in a simple manner to give a ready-to-use solution which contains no visible particles by addition of an aqueous solvent, for example water for injection purposes or an isotonic aqueous solution. The reconstituted solution is stable over a period of about 5 days, but is particularly preferably applied within four hours.

Reconstitution of the preparation according to the invention with aqueous solvents advantageously enables the preparation of antibody-containing solutions having a pH of from 5 to 8, preferably having a pH of from 6.0 to 7.4, particularly preferably having a pH of about 7.2, and an osmolality of from 250 to 350 mOsmol/kg. The reconstituted preparation can thus be administered directly intravenously, intraarterially and also subcutaneously substantially without pain. In addition, the preparation can also be added to infusion solutions, such as, for example, glucose solution, isotonic saline solution or Ringer solution, which may also comprise further active ingredients, thus also enabling relatively large amounts of active ingredient to be administered.

According to a preferred embodiment of the invention, the lyophilised pharmaceutical preparation essentially consists of an antibody, a sugar or amino sugar, an amino acid, a buffer and a surfactant.

The preparation according to the invention enables the preparation of antibody solutions which are matched in their concentration to the clinical needs. Preference is given to antibody solutions having an antibody concentration of from about 0.5 to 25 mg/ml, particularly preferably from 5 to 20 mg/ml, very particularly preferably from 10 to 15 mg/ml. The preparation according to the invention thus enables the preparation of ready-to-use preparations having a significantly higher antibody concentration than is described for the preparations of WO 98/22136 A2.

The sugar employed in the preparation according to the invention can be a mono-, di- or trisaccharide. Examples of monosaccharides that may be mentioned are glucose, mannose, galactose, fructose and sorbose, examples of disaccharides that may be mentioned are sucrose, lactose, maltose and trehalose, and an example of a trisaccharide that may be mentioned is raffinose. Preference is given to sucrose, lactose, maltose and trehalose, particularly preferably sucrose.

It is also possible for amino sugars to be present, i.e. monosaccharides which contain a primary, secondary or tertiary amino group or an acylated amino group (—NH—CO—R) instead of a hydroxyl group. For the purposes of the invention, particular preference is given here to glucosamine, N-methylglucosamine, galactosamine and neuraminic acid.

The sugar/amino sugar is present in the preparation according to the invention in such an amount that it is present in the resultant solution after reconstitution with the proposed volume of solvent in a concentration of from about 1 to 200 mg/ml. The sugar is preferably present in the reconstituted solution in a concentration of from 30 to 65 mg/ml.

Suitable amino acids used in accordance with the invention are basic amino acids, such as, for example, arginine, histidine, ornithine, lysine, inter alia, the amino acids preferably being employed in the form of their inorganic salts (advantageously in the form of the hydrochloric acid salts, i.e. as amino acid hydrochlorides). In the case where the free amino acids are employed, the desired pH is set by addition of a suitable physiologically tolerated buffer substance, such as, for example, an organic or inorganic acid, such as citric acid or phosphoric acid, sulfuric acid, acetic acid, formic acid or salts thereof. Preference is given to citrates and phosphates, with which particularly stable lyophilisates are obtained.

Preferred amino acids are arginine, lysine and ornithine. In addition, it is also possible to use acidic amino acids, such as, for example, glutamic acid and aspartic acid, or neutral amino acids, such as, for example, isoleucine, leucine and alanine, or aromatic amino acids, such as, for example, phenylalanine, tyrosine or tryptophan. The amino acid content in the preparation according to the invention is from 1 to 100 mg/ml, preferably from 1 to 50 mg/ml, particularly preferably 3-30 mg/ml (in each case based on the reconstituted solution).

Surfactants which can be employed are all surfactants usually used in pharmaceutical preparations, preferably polysorbates and polyoxyethylene-polyoxypropylene polymers. Particular preference is given to polyoxyethylene (20) sorbitan monolaurate and polyoxyethylene (20) sorbitan monooleate. In accordance with the invention, the preparation comprises from 0.001 to 1% by weight, preferably from 0.005 to 0.1% by weight and particularly preferably about 0.01% by weight (in each case based on the reconstituted solution).

If the preparation according to the invention comprises buffers, these can in principle be any physiologically tolerated substances which are suitable for setting the desired pH. The amount of buffer substance is selected in such a way that, after reconstitution of the lyophilised preparation, for example with water for injection purposes, the resultant aqueous solution has a buffer concentration of from 5 mmol/l to 20 mmol/l, preferably about 10 mmol/l. Preferred buffers are citrate buffers or phosphate buffers. Suitable phosphate buffers are solutions of mono- and/or disodium and -potassium salts of phosphoric acid, such as disodium hydrogenphosphate or potassium dihydrogenphosphate, as well as mixtures of the sodium and potassium salts, such as, for example, mixtures of disodium hydrogenphosphate and potassium dihydrogenphosphate.

If the reconstituted solution is not already isotonic through the osmotic properties of the antibody and the auxiliaries employed for stabilisation, an isotonic agent, preferably a physiologically tolerated salt, such as, for example, sodium chloride or potassium chloride, or a physiologically tolerated polyol, such as, for example, glucose or glycerol, may furthermore be present in a concentration necessary for establishing isotonicity.

In addition, the lyophilisates according to the invention may comprise further physiologically tolerated auxiliaries, such as, for example, antioxidants, such as ascorbic acid or glutathione, preservatives, such as phenol, m-cresol, methyl- or propylparaben, chlorobutanol, thiomersal or benzalkonium chloride, polyethylene glycols (PEG), such as PEG 3000, 3350, 4000 or 6000, or cyclodextrins, such as hydroxypropyl-β-cyclodextrin, sulfobutylethyl-β-cyclodextrin or γ-cyclodextrin.

The preparation according to the invention can be prepared by preparing an aqueous preparation comprising Cetuximab® or EMD 72000 as active ingredient and a sugar or amino sugar, an amino acid and a surfactant as additives and, if desired, further pharmaceutical auxiliaries, and subsequently lyophilising the solution.

The aqueous preparation can be prepared by adding the said auxiliaries to a solution comprising Cetuximab® or EMD 72000. To this end, defined volumes of stock solutions comprising the said further auxiliaries in defined concentration are advantageously added to a solution having a defined concentration of Cetuximab® or EMD 72000, as obtained from its preparation, and the mixture is, if desired, diluted to the pre-calculated concentration with water. Alternatively, the auxiliaries can also be added as solids to the starting solution comprising Cetuximab®. If Cetuximab® or EMD 72000 is in the form of a solid, for example in the form of a lyophilisate, the preparation according to the invention can be prepared by firstly dissolving the respective antibodies in water or an aqueous solution comprising one or more of the further auxiliaries, and subsequently adding the amounts required in each case of stock solutions comprising the further auxiliaries, the further auxiliaries in solid form and/or water. Cetuximab® or EMD 72000 can advantageously also be dissolved directly in a solution comprising all further auxiliaries.

One or more of the auxiliaries present in the preparation according to the invention may advantageously already have been added during or at the end of the process for the preparation of the particular EGFR antibody. This can preferably be carried out by dissolving Cetuximab® or EMD 72000 directly in an aqueous solution comprising one, more than one or all of the further auxiliaries in the final step of the purification carried out after its preparation. In order to prepare the preparation, the respective further ingredient(s) then need only be added in a smaller amount in each case and/or not added at all. It is particularly preferred for the respective ingredient to be dissolved directly in an aqueous solution comprising all further auxiliaries in the final step of the purification carried out after its preparation, directly giving the solution to be lyophilised.

The solution comprising the respective antibody and the auxiliaries is set to a pH of from 5 to 8, sterile-filtered and freeze-dried.

The examples explain the invention without being restricted thereto.

If 10 mmol/l of sodium phosphate buffer or potassium phosphate buffer pH 7.2 were employed, this comprised 2.07 g/l of disodium hydrogenphosphate 7-hydrate and 0.31 g/l of sodium dihydrogenphosphate monohydrate or 1.220 g/l of dipotassium hydrogenphosphate and 0.4050 g/l of potassium dihydrogenphosphate.

### EXAMPLE 1

Lyophilisate obtained from aqueous solution comprising:
10 mg/ml of EMD 72000
10 mmol/l of potassium phosphate buffer pH 7.2
17 mmol/l of arginine
3% by weight of sucrose
0.01% by weight of polyoxyethylene (20) sorbitan monolaurate
0.4% by weight of PEG 6000

The preparation was carried out by mixing defined volumes of aqueous solutions comprising the respective auxiliaries in defined concentration.

The following solutions were used:

Solution A (active-ingredient solution) comprising:
20 mg/ml of EMD 72000
10 mmol/l of potassium phosphate buffer pH 7.2

Solution B (buffer/salt solution):
10 mmol/l of potassium phosphate buffer pH 7.2
6% by weight of sucrose
0.02% by weight of polyoxyethylene (20) sorbitan monolaurate
34 mmol/I of arginine
0.8% by weight of polyethylene glycol 6000

In order to prepare the preparation according to the invention, equal volumes of solution A and solution B were combined with one another.

The prepared solution was sterile-filtered before packaging. The vials were each filled with 2 ml of solution. The vials were subsequently partially sealed with stoppers and lyophilised. After freeze-drying, the vials were sealed and crimped.

### EXAMPLE 2

Lyophilisate obtained from aqueous solution comprising:
10 mg/ml of EMD 72000
10 mmol/l of potassium phosphate buffer pH 7.2
14 mmol/l of arginine
3% by weight of sucrose
0.01% by weight of polyoxyethylene (20) sorbitan monolaurate

The preparation was carried out by mixing defined volumes of aqueous solutions comprising the respective auxiliaries in defined concentration.

The following solutions were used:

Solution A (active-ingredient solution) comprising:
20 mg/ml of EMD 72000
10 mmol/l of potassium phosphate buffer pH 7.2

Solution B (buffer/salt solution):
10 mmol/l of potassium phosphate buffer pH 7.2
6% by weight of sucrose
0.02% by weight of polyoxyethylene (20) sorbitan monolaurate
34 mmol/l of arginine

In order to prepare the preparation according to the invention, equal volumes of solution A and solution B were combined with one another.

The prepared solution was sterile-filtered before packaging. The vials were each filled with 20 ml of solution. The vials were subsequently partially sealed with stoppers and lyophilised. After freeze-drying, the vials were sealed and crimped.

### EXAMPLE 3

Lyophilisate obtained from aqueous solution comprising:
15 mg/ml of Cetuximab®
5 mmol/l of citrate
100 mmol/l of arginine
4% by weight of mannitol
0.01% by weight of polyoxyethylene (20) sorbitan monooleate

The preparation was carried out by mixing defined volumes of aqueous solutions comprising the respective auxiliaries in defined concentration.

The following solutions were used:

Solution A (active-ingredient solution) comprising:
19 mg/ml of Cetuximab®
5 mmol/l of citrate
127 mmol/l of arginine
0.01% by weight of polyoxyethylene (20) sorbitan monooleate

Solution B (buffer/salt solution):
5 mmol/l of citrate
19.05% by weight of mannitol
0.01% by weight of polyoxyethylene (20) sorbitan monooleate

In order to prepare the preparation according to the invention, 7.9 ml of solution A and 2.1 ml of solution B were combined with one another.

The prepared solution was filtered using a sterile filter before packaging. The vials were each filled with 2 ml of solution using a pipette. The vials were subsequently partially sealed with stoppers and lyophilised. After freeze-drying, the vials were sealed and crimped.

### EXAMPLE 4

Lyophilisate obtained from aqueous solution comprising:
15 mg/ml of Cetuximab®
5 mmol/l of citrate
100 mmol/l of arginine
1.5% by weight of sucrose
0.01% by weight of polyoxyethylene (20) sorbitan monolaurate

The preparation was carried out by mixing defined volumes of aqueous solutions comprising the respective auxiliaries in defined concentration.

The following solutions were used:

Solution A (active-ingredient solution) comprising:
19 mg/ml of Cetuximab®
5 mmol/l of citrate
127 mmol/l of arginine
0.01% by weight of polyoxyethylene (20) sorbitan monooleate

Solution B (buffer/salt solution):
5 mmol/l of citrate
7.1% of sucrose
0.01% by weight of polyoxyethylene (20) sorbitan monooleate

In order to prepare the preparation according to the invention, 7.9 ml of solution A and 2.1 ml of solution B were combined with one another.

The prepared solution was filtered using a sterile filter before packaging. The vials were each filled with 2 ml of solution using a pipette. The vials were subsequently partially sealed with stoppers and lyophilised. After freeze-drying, the vials were sealed and crimped.

### EXAMPLE 5

Lyophilisate obtained from aqueous solution comprising:
15 mg/ml of Cetuximab®
10 mmol/l of potassium phosphate buffer pH 7.2
14 mmol/l of L-arginine hydrochloride
88 mmol/l of sucrose
0.01% by weight of polyoxyethylene (20) sorbitan monolaurate adjust to pH 7.5 using phosphoric acid

The preparation was carried out by mixing defined volumes of aqueous solutions comprising the respective auxiliaries in defined concentration.

The following solutions were used:

Solution A (active-ingredient solution) comprising:
25 mg/ml of Cetuximab®
10 mmol/l of potassium phosphate buffer pH 7.2 water for injection purposes

Sultion B (buffer/salt solution):
10 mmol/l of potassium phosphate buffer pH 7.2
35.6 mmol/l of L-arginine hydrochloride
219 mmol/l of sucrose
0.025% by weight of polyoxyethylene (20) sorbitan monolaurate adjust to pH 7.5 using phosphoric acid

In order to prepare the preparation according to the invention, 6 ml of solution A and 4 ml of solution B were combined with one another.

The prepared solution was filtered using a sterile filter before packaging. The vials were each filled with 2 ml of solution using a pipette. The vials were subsequently partially sealed with stoppers and lyophilised. After freeze-drying, the vials were sealed and crimped.

### EXAMPLE 6

### Comparative Preparation 1

Aqueous solution comprising:
5 mg/ml of Cetuximab®
10 mmol/l of sodium phosphate buffer pH 7.2
145 mmol/l of sodium chloride

The preparation was carried out by mixing defined volumes of aqueous solutions comprising the respective auxiliaries in defined concentration.

The following solutions were used:

Solution A (active-ingredient solution) comprising:
10 mg/ml of Cetuximab®
10 mmol/l of sodium phosphate buffer pH 7.2
145 mmol/l of sodium chloride

(The solution was obtained by eluting the active ingredient from the column using solution B in the final step of the chromatographic active-ingredient purification carried out after its preparation.)

Solution B (buffer/salt solution):

Corresponds to solution A, but comprises no active ingredient.

In order to prepare the comparative preparation, 10 ml of each of solutions A and B were combined with one another.

### EXAMPLE 7

### Comparative Preparation 2

Aqueous solution comprising:
5 mg/ml of Cetuximab@
10 mmol/l of sodium phosphate buffer pH 7.2
45 mmol/l of sodium chloride
0.01% by weight of polyoxyethylene (20) sorbitan monooleate

The preparation was carried out by mixing defined volumes of aqueous solutions comprising the respective auxiliaries in defined concentration.

The following solutions were used:

Solution A (active-ingredient solution) comprising:
9.7 mg/ml of Cetuximab®
10 mmol/l of sodium phosphate buffer pH 7.2
145 mmol/l of sodium chloride

(The solution was obtained by eluting the active ingredient from the column using solution B in the final step of the chromatographic active-ingredient purification carried out after its preparation.)

Solution B (buffer/salt solution):

Corresponds to solution A, but comprises no active ingredient.

Solution C (polyoxyethylene sorbitan fatty acid ester solution):

Corresponds to solution B, but additionally comprises 1% by weight of polyoxyethylene (20) sorbitan monooleate.

In order to prepare the comparative preparation, 10 ml of solution A, 9.8 ml of solution B and 0.2 ml of solution C were combined with one another.

The prepared solution was filtered using a sterile filter before packaging. The vials were each filled with 2 ml of solution using a pipette. The vials were subsequently sealed with stoppers and crimped.

Investigations of the Stability of the Preparations

The stability of the preparations according to the invention from Examples 1 and 2 was tested in stress tests. To this end, the lyophilisates prepared were stored at 40° C. and a relative atmospheric humidity (RAH) of 75%. The preparations were stored for certain times and analysed using suitable analytical methods. Possible instabilities were evident in the antibodies principally from the formation of aggregates and from the formation of degradation products. Degradation products are preferably detected by gel electrophoresis (sodium dodecylsulfate/polyacrylamide gel electrophoresis (SDS-PAGE) and isoelectric focusing (IEF)), whereas visual inspection and turbidity measurements were used to detect visible aggregates and size exclusion chromatography (HPLC-SEC) was used to detect soluble aggregates. The ELISA (enzyme linked immunosorbent assay) test likewise used for evaluating the preparations serves to check the integrity and binding ability to the receptor.

Results in Tables 1 and 2 clearly confirm the quality and stability of the preparations prepared, even at elevated storage temperatures and elevated relative atmospheric humidity (40° C./75% RAH).

TABLE 1
Stability of the preparation from Example 1

| | | | SDS-PAGE | | |
|---|---|---|---|---|---|
| | | | [% of IG monomer] | | |
| Storage time | | ELISA | non- | | |
| (months) | pH | (rel. titre) | reducing | reducing | SE-HPLC |

Temperature 2-8° C.

| | | | | | |
|---|---|---|---|---|---|
| 0 | 7.11 | 1.00 | 100 | 100 | 100 |
| 6 | 7.14 | 1.01 | 99.3 | 100 | 99.6 |
| 12 | 7.15 | 1.04 | 99.9 | 99.1 | 99.7 |
| 24 | 7.16 | 1.03 | 100 | 100 | 100 |

Temperature 25° C., 60% rel. humidity

| | | | | | |
|---|---|---|---|---|---|
| 6 | 7.17 | 1.03 | 99.1 | 99.0 | 99.5 |
| 12 | 7.15 | 1.01 | 98.7 | 99.1 | 99.6 |
| 24 | 7.16 | 1.03 | 100 | 100 | 99.5 |

Temperature 40° C., 75% rel. humidity

| | | | | | |
|---|---|---|---|---|---|
| 6 | 7.17 | 1.10 | 98.8 | 98.2 | 99.3 |
| 12 | 7.15 | n.m. | n.m. | n.m. | n.m. |
| 24 | 7.16 | 0.98 | 100 | 100 | 99.3 |

TABLE 2
Stability of the preparation from Example 2

| | | | SDS-PAGE | | |
|---|---|---|---|---|---|
| | | | [% of IG monomer] | | |
| Storage time | | ELISA | non- | | |
| (weeks) | pH | (rel. titre) | reducing | reducing | SE-HPLC |

Temperature 2-8° C.

| | | | | | |
|---|---|---|---|---|---|
| 0 | 7.20 | 0.97 | 100 | 100 | 99.33 |
| 13 | 7.23 | 1.00 | n.m. | n.m. | 99.24 |
| 26 | 7.21 | 1.02 | 99.47 | 99.57 | 99.27 |

Temperature 25° C., 60% rel. humidity

| | | | | | |
|---|---|---|---|---|---|
| 13 | 7.24 | 1.05 | n.m. | n.m. | 99.22 |
| 26 | 7.24 | 0.97 | 99.47 | 99.64 | 99.21 |

Temperature 40° C., 75% rel. humidity

| | | | | | |
|---|---|---|---|---|---|
| 13 | 7.23 | 1.04 | n.m. | n.m. | 99.08 |
| 26 | 7.25 | 0.97 | 99.61 | 99.62 | 98.85 |

The stability of the preparations according to the invention from Examples 3 to 7 were likewise checked in stress tests. To this end, vials containing the solution from Examples 3-5 and, for comparative purposes, vials containing the solution from Examples 6-7 were stored at 25° C. and 60% relative atmospheric humidity (RAH) and 40° C. and 75% RAH. Before storage and after defined storage times, in each case 3 vials were assessed visually under direct illumination with a cold light source, and the absorption of the solutions at 350 and 550 nm, which represents a measure of the turbidity, was determined. Furthermore, 3 vials were removed in each case and analysed with regard to the content of Cetuximab® and decomposition products by means of HPLC gel filtration.

The HPLC chromatographic studies were carried out with acetonitrile/water 95/5 (V/V) gradients (B) and buffer solution pH 2.5/acetonitrile 95/5 (V/V) (A) as eluents. Column: LiChroCHAR® 125-2 HPLC cartridge; Super-spher® 60 RP-select B, flow rate: 0.3 ml/min, detection at 210 nm.

The results of the stability studies are shown in Table 3.

TABLE 3

| | Storage at | | | | | | |
|---|---|---|---|---|---|---|---|
| | 40° C./75% | | | Decomposition | Turbidity | Turbidity | |
| Test | RAH | Cetuximab | Aggregates | products | at λ = | at λ = | Visual |
| solution | [weeks] | [%] | [%] | [%] | 350 nm | 550 nm | assessment |
| Ex. 3 | 0 | 99.30 | 0.20 | 0.51 | 0.0211 | 0.0055 | clear |
| Ex. 3 | 4 | 99.04 | 0.62 | 0.34 | 0.0214 | 0.0029 | clear |
| Ex. 3 | 8 | 98.54 | 1.12 | 0.34 | 0.0224 | 0.0020 | clear |
| Ex. 3 | 13 | 98.43 | 1.18 | 0.40 | 0.0246 | 0.0019 | clear |
| Ex. 4 | 0 | 99.33 | 0.19 | 0.48 | 0.0198 | 0.0045 | clear |
| Ex. 4 | 4 | 99.11 | 0.40 | 0.50 | 0.0174 | 0.0025 | clear |
| Ex. 4 | 8 | 99.19 | 0.44 | 0.38 | 0.0181 | 0.0021 | clear |
| Ex. 4 | 13 | 99.20 | 0.45 | 0.36 | 0.0172 | 0.0014 | clear |
| Ex. 5 | 0 | 99.58 | 0.19 | 0.04 | 0.0195 | 0.0050 | clear |
| Ex. 5 | 4 | 99.51 | 0.24 | 0.25 | 0.0165 | 0.0028 | clear |
| Ex. 5 | 8 | 99.56 | 0.22 | 0.22 | 0.0156 | 0.0023 | clear |
| Ex. 5 | 13 | 99.52 | 0.28 | 0.20 | 0.0187 | 0.0045 | clear |
| Ex. 6 | 0 | 99.69 | 0.62 | 0.15 | 0.0130 | 0.0021 | clear |
| Ex. 6 | 4 | 92.00 | 0.84 | 7.38 | 0.0232 | 0.0047 | cloudy |
| Ex. 6 | 8 | 89.94 | 1.39 | 8.68 | 0.0338 | 0.0044 | cloudy |
| Ex. 6 | 13 | 86.66 | 3.26 | 10.11 | 0.0403 | 0.0061 | cloudy |
| Ex. 7 | 0 | 99.72 | 0.17 | 0.11 | 0.0128 | 0.0016 | clear |
| Ex. 7 | 4 | 98.60 | 0.56 | 0.84 | 0.0200 | 0.0022 | clear |
| Ex. 7 | 8 | 96.49 | 2.21 | 1.32 | 0.0280 | 0.0033 | clear |
| Ex. 7 | 13 | 86.46 | 4.84 | 8.73 | 0.0382 | 0.0036 | cloudy |

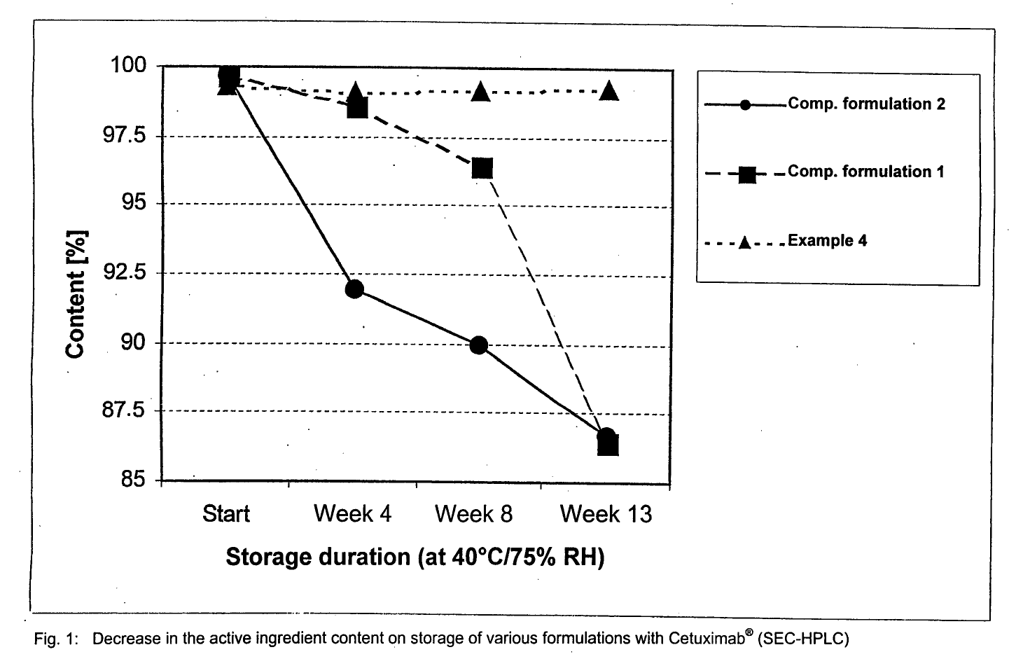

FIGS. 1 to 5 furthermore show a comparison of the results of various stability studies of the preparation according to the invention from Example 4 with comparative formulations 1 and 2 after defined storage times at 40° C. and 75% RAH. Before each analysis was carried out, the freeze-dried preparation from Example 4 was reconstituted with water for injection purposes to give an aqueous solution containing three times the amount of water compared with the starting solution used to prepare the lyophilised preparation by freeze-drying.

FIG. 1 shows the decrease in the active-ingredient content in comparative formulations 1 and 2 compared with the preparation according to the invention from Example 4, measured as the content of monomer in the HPLC-SEC.

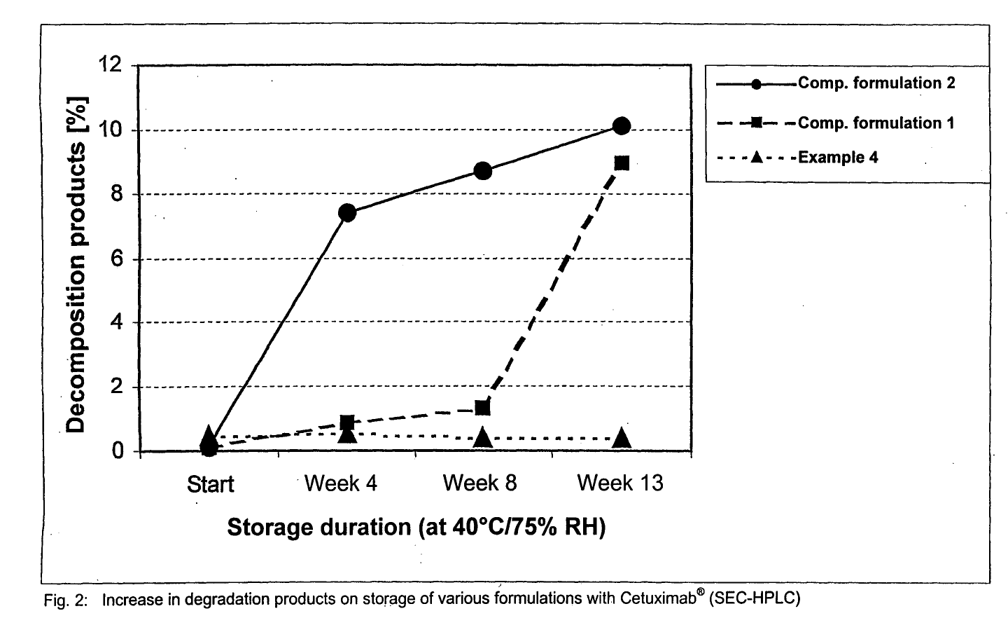

FIG. 2 shows the increase in degradation products in comparative formulations 1 and 2 compared with the preparation according to the invention from Example 4, measured in the HPLC-SEC.

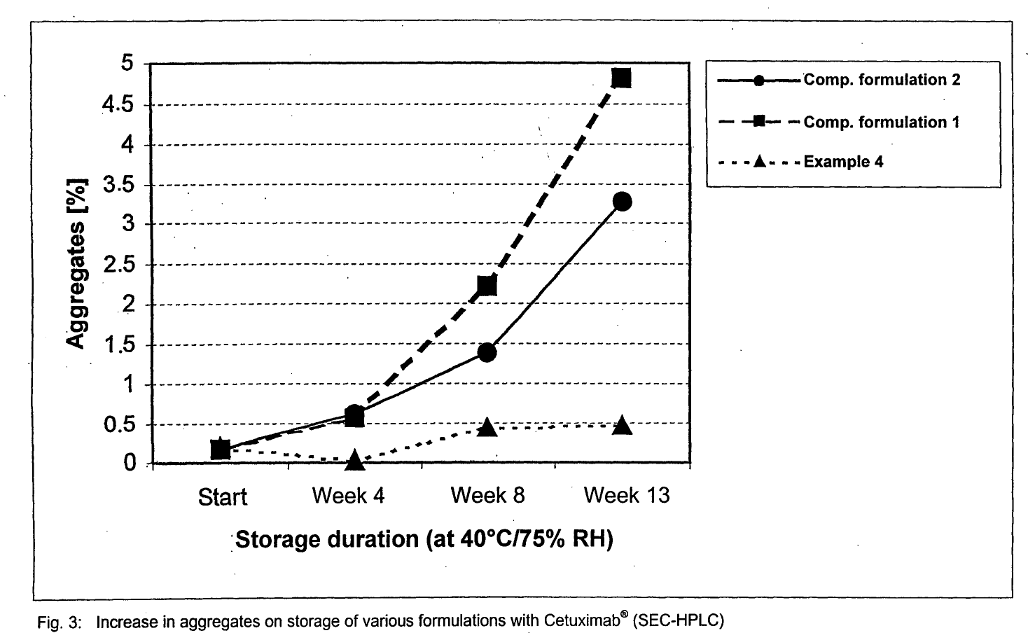

FIG. 3 shows the increase in aggregates in comparative formulations 1 and 2 compared with the preparation according to the invention from Example 4, measured in the HPLC-SEC.

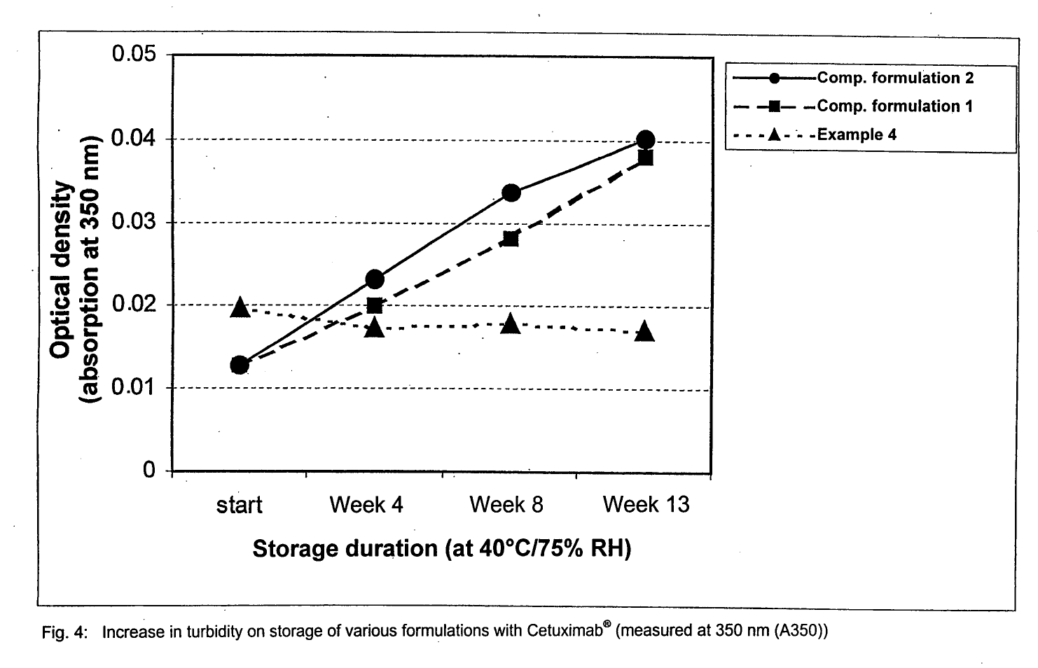

FIG. 4 shows the increase in optical density at λ=350 nm in comparative formulations 1 and 2 compared with the preparation according to the invention from Example 4.

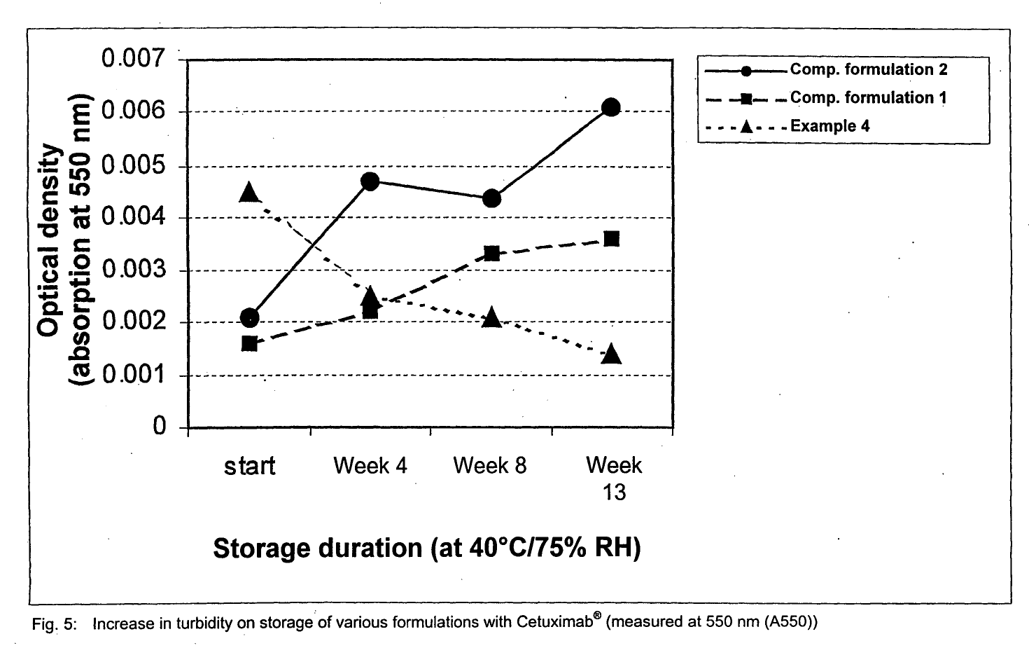

FIG. 5 shows the increase in optical density at λ=550 nm in comparative formulations 1 and 2 compared with the preparation according to the invention from Example 4.

The results clearly show that the compositions according to the invention have significantly increased stability compared with the liquid comparative solutions.

1. Lyophilised pharmaceutical preparation of mono- or polyclonal antibodies comprising a sugar or an amino sugar, an amino acid and a surfactant, characterised in that the antibody is an antibody directed against the epidermal growth factor receptor (EGF receptor).

2. Lyophilised pharmaceutical preparation according to claim 1, characterised in that the antibody is Cetuximab® or EMD 72000 or one of the murine, humanised or chimeric antibody analogues corresponding thereto.

3. Lyophilised pharmaceutical preparation according to claim 2, characterised in that the antibody is Cetuximab® or EMD 72000.

4. Lyophilised pharmaceutical preparation according to claim 1, essentially consisting of an antibody, a sugar or amino sugar, an amino acid, a buffer and a surfactant.

5. Lyophilised pharmaceutical preparation according to claim 1, characterised in that the sugar is a mono-, di- or trisaccharide, preferably sucrose, maltose or trehalose.

6. Lyophilised pharmaceutical preparation according to claim 1, characterised in that the amino sugar is glucosamine, N-methylglucosamine, galactosamine or neuraminic acid.

7. Lyophilised pharmaceutical preparation according to claim 1, characterised in that the amino acid is a basic, acidic or neutral amino acid, preferably arginine, lysine or ornithine.

8. Lyophilised pharmaceutical preparation according to claim 1, characterised in that the surfactant is a polysorbate or a polyoxyethylene-polyoxypropylene polymer.

9. Lyophilised pharmaceutical preparation according to claim 8, characterised in that the surfactant is the polyoxyethylene sorbitan fatty acid ester polyoxyethylene (20) sorbitan monooleate or polyoxyethylene (20) sorbitan monolaurate.

10. Lyophilised pharmaceutical preparation according to claim 1, characterised in that an isotonic agent in a concentration necessary for establishing isotonicity is furthermore present.

11. Lyophilised pharmaceutical preparation according to claim 10, characterised in that sodium chloride is present as isotonic agent.

12. Aqueous pharmaceutical preparation of mono- or polyclonal antibodies obtainable by reconstituting the lyophilisate according to claim 1 with an aqueous solvent.

13. Aqueous pharmaceutical preparation according to claim 12, characterised in that the solution has a pH of 5-8, preferably 6-7.4.

14. Aqueous pharmaceutical preparation according to claim 13, characterised in that the solution has a pH of about 7.2.

15. Process for the preparation of a lyophilised pharmaceutical preparation according to claim 1, characterised in that an aqueous preparation comprising Cetuximab® or EMD 72000 as active ingredient and a sugar or amino sugar, an amino acid and a surfactant as additives and, if desired, further pharmaceutical auxiliaries, is prepared, and the solution is subsequently lyophilised.
